(19) United States
(12) Patent Application Publication
(10) Pub. No.: US 2002/0072791 A1
(43) Pub. Date: Jun. 13, 2002

(54) Light-activated multi-point detachment mechanism

(76) Inventors: Joseph C. Eder, Los Altos Hills, CA (US); Robert M. Abrams, Encinitas, CA (US)

Correspondence Address: ROBINS & PASTERNAK LLP, 545 MIDDLEFIELD ROAD, SUITE 180, MENLO PARK, CA, 94025, US

(21) Appl. No.: 09/733,515

(22) Filed: Dec. 7, 2000

(51) Int. Cl. A61M 29/00

(52) U.S. Cl. 623/1.15; 606/15; 606/33; 606/157; 606/200

(57) ABSTRACT

Implantable devices having multiple detachment junctions are disclosed. Each junction is activated by a different wavelength of electromagnetic radiation, for example light. Also described are methods of making and using these devices.

### FIELD OF THE INVENTION

This invention relates to the field of implantable devices. More particularly, it relates to implantable devices having multiple detachment junctions. Each detachment junction is activated by a unique wavelength of electromagnetic radiation (e.g, light).

### BACKGROUND

There are a variety of implantable devices that require precise placement within the vasculature of the human body. Such devices include vaso-occlusive coils, stents, filters and other three-dimensional devices. Vaso-occlusive coils are described, for example, in U.S. Pat. No. 4,994,069, to Ritchart et al.; U.S. Pat. No. 5,624,461 to Mariant; U.S. Pat. No. 5,639,277 to Mariant et al. and U.S. Pat. No. 5,649,949 to Wallace et al. describes variable cross-section conical vaso-occlusive coils. Stents are described, for example, in U.S. Pat. No. 4,655,771 to Wallsten; U.S. Pat. No. 4,954,126 to Wallsten and U.S. Pat. No. 5,061,275 to Wallsten et al.

Typically, implantable devices include a single detachment mechanism in order to be released from the deployment mechanism (e.g., attached wire). One class of detachment mechanisms involves the use of electrolytic means to detach the vaso-occlusive member from the pusher. In one technique (U.S. Pat. No. 5,122,136 to Guglielmi et al.) the vaso-occlusive member is bonded via a metal-to-metal joint to the distal end of the pusher. The pusher and vaso-occlusive member are made of dissimilar metals. The vaso-occlusive member-carrying pusher is advanced through the catheter to the site and a low electrical current is passed through the pusher-vaso-occlusive member assembly. The current causes the joint between the pusher and the vaso-occlusive member to be severed via electrolysis. The pusher may then be retracted leaving the detached vaso-occlusive member at an exact position within the vessel. In addition to enabling more accurate vaso-occlusive member placement, the electric current may facilitate thrombus formation at the vaso-occlusive member site. The only perceived disadvantage of this method is that the electrolytic release of the vaso-occlusive member requires a period of time so that rapid detachment of the vaso-occlusive member from the pusher does not occur. Other examples of this technique can be found in U.S. Pat. No. 5,423,829 to Pham et al. and U.S. Pat. No. 5,522,836 to Palermo.

Other forms of energy are also used to sever sacrificial joints that connect pusher and vaso-occlusive member apparatus. An example is that shown in Japanese Laid-Open Patent Application No. 7-265431 or corresponding U.S. Pat. No. 5,759,161 and U.S. Pat. No. 5,846,210 to Ogawa et al. A sacrificial connection member, preferably made from polyvinylacetate (PVA), resins, or shape memory alloys, joins a conductive wire to a detention member. Upon heating by a monopolar high frequency current, the sacrificial connection member melts, severing the wire from the detention member. U.S. Pat. No. 5,944,733 to Engelson describes application of radio-frequency energy to sever a thermoplastic joint.

In U.S. Pat. No. 4,735,201 to O'Reilly, an optical fiber is enclosed within a catheter and connected to a metallic tip on its distal end by a layer of hot-melt adhesive. The proximal end of the optical fiber is connected to a laser energy source. When endovascularly introduced into an aneurysm, laser energy is applied to the optical fiber, heating the metallic tip so as to cauterize the immediately surrounding tissue. The layer of hot-melt adhesive serving as the bonding material for the optical fiber and metallic tip is melted during this lasing, but the integrity of the interface is maintained by application of back pressure on the catheter by the physician. When it is apparent that the proper therapeutic effect has been accomplished, another pulse of laser energy is then applied to once again melt the hot-melt adhesive, but upon this reheating the optical fiber and catheter are withdrawn by the physician, leaving the metallic tip in the aneurysm as a permanent plug.

Other methods for placing implantable devices within the vasculature utilize heat releasable bonds that can be detached by using laser energy (see, U.S. Pat. No. 6,102,917). EP 0 992 220 describes an embolic coil placement system which includes conductive wires running through the delivery member. When these wires generate sufficient heat, they are able to sever the link between the embolic coil and the delivery wires. Further, U.S. Ser. No. 09/177,848 describes the use of fluid pressure (e.g., hydraulics) to detach an embolic coil.

None of these documents disclose devices having multiple detachment points, each of which is detachable by applying a different wavelength of electromagnetic radiation.

### SUMMARY OF THE INVENTION

The present invention includes implantable devices having multiple detachment points. Each detachment junction can be severed using a different wavelength of electromagnetic radiation, e.g., light.

Thus, in one aspect, the invention includes an implantable device comprising a plurality of detachment junctions, wherein each junction is cleaved by the application of a different wavelength of electromagnetic radiation. In certain embodiments, the electromagnetic radiation is light, for example visible light or non-visible light. In other embodiments, one or more of the plurality of detachment junctions comprise a shape memory polymer and/or one or more pigments or dyes. The implantable device can be any device, for example, a vaso-occlusive coil, a stent, a filter, or the like.

In another aspect, the invention includes an assembly for use in delivering an implantable device comprising (a) an implantable device according to claim 1; and (b) a deployment mechanism. In certain embodiments, the deployment mechanism comprises one or more electromagnetic radiation transmitting devices, for example one or more fiber optic cables; one or more light-transmitting fluids; one or more light-transmitting wires; or the like. The implantable device can be, for example, a vaso-occlusive coil, a stent, a filter or the like. In various embodiments, the assemblies described herein further include a source of electromagnetic radiation attached to the delivery mechanism, for example a light source (e.g., laser).

These and other embodiments of the subject invention will readily occur to those of skill in the art in light of the disclosure herein.

### DESCRIPTION OF THE INVENTION

Implantable devices, such as vaso-occlusive coils or stents, are described. The devices include multiple detachment points, wherein each detachment point is activated (e.g., detached) by application of a different wavelength of electromagnetic radiation (e.g., light). In this way, the operator can select the desired point of detachment and/or the order in which the device is deployed. Methods of making and using these devices also form an aspect of this invention.

Advantages of the present invention include, but are not limited to, (i) increasing the precision of placement of implantable devices; (ii) increasing the speed at which implantable devices can be deployed; (iii) providing vaso-occlusive devices that are more precisely sized for the desired purpose; and (iv) providing methods and materials for making these multi-detachment junction devices.

All publications, patents and patent applications cited herein, whether supra or infra, are hereby incorporated by reference in their entirety.

It must be noted that, as used in this specification and the appended claims, the singular forms “a”, “an”, and “the” include plural referents unless the content clearly dictates otherwise. Thus, for example, reference to “a implantable device” includes a two or more such devices.

The present invention is directed to implantable devices which contain more than one detachment site. Further, each detachment site (or junction) is light-activated (e.g., photo-cleavable) and, further, each of these multiple detachment sites (or junctions) are detached using different wavelengths of light. No limitation is set on the nature of the material making up the junction, so long as it is not cytotoxic and is cleavable by the application of electromagnetic radiation. As will be apparent to those of skill in the art, the junction need not melt completely in order to be severable from the implantable device. Rather, the junction need only melt sufficiently that the operator can remove the delivery mechanism.

In preferred embodiments, the detachment sites are made up of one or more shape memory polymers which are known to change physical properties with temperature and, in addition, have low cytotoxicity. See, e.g., U.S. Pat. Nos. 6,102,917; 6,086,599; 6,102,933. In response to changes in temperature, these shape memory polymers change their physical properties include hardness, flexibility, modulus of elasticity and shape. Warming followed by cooling allows forming of the material; the new shape is retained until the part is rewarmed and re-cooled, at which time the part reverts to its original manufactured shape. Such polymers can be readily designed and manufactured such that they preferentially absorb electromagnetic radiation (e.g., light) of a specific range of wavelengths. The electromagnetic (e.g, light) energy absorbed by the polymer is then converted into heat energy which in turn melts the polymer and allows for detachment at that site.

Shape memory polymers that respond preferentially to a specific wavelength of energy (or to a relatively narrow range of wavelengths) are known and can be readily manufactured using commercially available materials. See, e.g., U.S. Pat. Nos. 6,102,917; 6,102,933 and 6,086,599 for a discussion of shape memory polymers and using these polymers to form connections. Examples of other thermoplastics that may be used singly or in combination include, but are not limited to, materials such as polyactide, polyglycolide, polyactide-co-glycolide polydioxanone, polyethylene, polyiminocarbonates, polycaprolactone, polyesters and the like. U.S. Pat. No. 5,292,321 to Lee discusses such suitable thermoplastic materials. Additionally, suitable non-toxic pigments or dyes which affect light adsorption can also be added to the material making up the junction to achieve the desired responsive of the junction to a specific wavelength or range of wavelengths.

Any wavelength of electromagnetic radiation (e.g., light) is suitable for use in the present invention, so long as the amounts and duration of exposure to the energy source is not detrimental to the subject. The visible light spectrum extends from the low-energy red at approximately 7000 Å to the high-energy violet at approximately 4000 Å. Further, non-visible light wavelengths may also be used, for example, gamma rays; ultra-violet light (ranging from about 4000 Å to about 600 Å in wavelength and about 10 eV in energy); infra-red (ranging from about 7000 Å to 1 mm in wavelength and 10⁻³ eV to about 1 eV in energy); microwaves (ranging from about 1 mm to 3 cm in wavelength and from about 10⁻⁵ eV to 0.001 eV in energy); ultrahigh frequence (UHF, ranging from about 10⁻⁷ eV to 10⁻⁵ eV in energy) and radio waves (ranging from about 10⁻¹² eV to about 10⁻⁸ eV in energy). Thus, the range of wavelengths of light that each detachment junction responds preferentially to will vary according to the type of light. Junctions that are detachable using visible light, for example, may preferentially respond to wavelengths in an approximately a 1000 Å range, more preferably wavelengths within about a 500 Å range and even more preferably wavelengths within about a 100 Å range. Additionally, one or more of the junctions may respond to visible light while other may respond to non-visible light.

The implantable devices including multiple detachment points may be manufactured by any method known in the art, for example, by casting, extruding, injection molding and solution coating. The sites of these junctions can be determined during manufacture based on the desired use of the implant and the desired final, deployed configuration. Thus, in certain embodiments, the implant is constructed such that the junctions member are spaced along the length of the implant to allow for precise sizing of the implant by detaching the device at the appropriate junction. The spacing of the junctions can further be determined based on the ultimate use of the implant. For example, if the implant comprises a vaso-occlusive device designed to be deployed within an aneurysm, the device can be constructed such that light-activated junctions are disposed in series along the length of the device. In this way, the operator can position the device in the aneurysm and detach it such that the desired length is deployed.

Alternatively, the multiple detachment junctions can be used in implantable devices where multiple detachment (or anchoring) points must be separated but where is desirable to perform each separation in a certain sequence. For example, in certain embodiments, an implantable device such as a stent will include multiple light-activated detachment points designed to be detached in a sequence determined by the operator. Thus, the type and location of each detachment junction can be selected on the basis of operator preference and ease of use.

Similarly, multiple detachment points can be used to more precisely configure an implantable device (e.g., a coil or a stent), for example, by detaching each appropriate junction as the distal end of the device forms the desired configuration, for example, pitch and spacing of a tubular coil structure.

Further, it will also be apparent that each detachment junction can be used to retrieve the devices from the vasculature, for example for removal or repositioning. Attachment of a single shape memory polymer junction to a guidewire or catheter are described for example in U.S. Pat. No. 6,086,599. However, the multiple, differentially light activated detachment mechanisms described herein allow for much more flexibility in both deployment and retrieval than single junctions. In particular, devices which include multiple detachment points can likewise be retrieved at any of those junctions by introducing a retrieval device with a known light-activated junction, positioning the retrieval device at the selected position on the device, and using the appropriate wavelength of light to reconnect the implantable device to the retrieval device.

The detachable junctions may be of a variety of thicknesses and coverage configurations depending upon a number of factors such as the type of implant, the degree of control over the release of the implantable device into the selected site desired by the user, the types and combinations of materials used, dimensional constraints of the catheter and sheath, and so forth. Typically, the diameter of each junction is between about 0.1-0.5 mm and the length anywhere from about 1 to 10 mm. For all configurations, it is desired that the thermoplastic member have a thickness that will not prohibit the engaged junctions from freely moving within a catheter sheath or other associated equipment necessary to accomplish the desired objective of reliably and safely placing a implantable device at a selected site.

One or more sources of electromagnetic radiation are connected to the junction member, for example via the delivery mechanism (e.g., wire). Preferably, a single source of energy that can be controlled by the operator to emit certain wavelengths of light is used. Alternatively, multiple sources of energy, each emitting different wavelengths corresponding to the preferentially absorption wavelengths of each junction, are used. Both fixed and variable sources of light, for example lasers, are known to those of skill in the art. In certain embodiments, one or more electromagnetic radiation transmitting devices (including for example, fiber optic cables, light-transmitting fluids, wires, etc. or combinations thereof) run through the delivery mechanism. These and other devices will be known to those of skill in the field.

A wide variety of implantable device comprising multiple differentially activated junctions can be designed and manufactured according to the teachings herein. The implant is desirably made up of a radiopaque, physiologically compatible material. For instance, the material may be platinum, gold, tungsten, or alloys of these. Certain polymers are also suitable for use in the implants, either alone or in conjunction with metallic markers providing radiopacity. These materials are chosen so that the procedure of locating the implant within the vessel may be viewed using radiography. However, it is also contemplated that the implantable device may be made of various other biologically inert polymers or of carbon fiber.

When the implantable member is a vaso-occlusive device such as a coil, its shape and constituent winding will depend upon the use to which the coil will be placed. For occluding peripheral or neural sites, the coils will typically be made of 0.05 to 0.15 mm diameter wire (platinum or platinum/tungsten alloy) that may be wound to have an inner diameter of 0.15 to 1.5 mm with a minimum pitch—that is to say that the pitch is equal to the diameter of the wire used in the coil. The outer diameter is then typically between 0.25 mm to 1.8 mm. The length of the coil will normally be in the range of 0.5 to 60 cm, preferably 0.5 to 40 cm. A discussion of this variation may be found, for example, in U.S. Pat. No. 4,994,069 to Ritchart et al. As noted above, light-activated junctions can be readily disposed along the length of the coil.

Conventional catheter insertion and navigational techniques involving guidewires or flow-directed devices may be used to access the site with a catheter. Briefly, the implantable devices having cleavable (e.g., photo-cleavable) detachable junctions described herein are typically loaded into a carrier for introduction into the delivery catheter and introduced to the chosen site using the procedure outlined below. This procedure may be used in treating a variety of maladies. For instance, in treatment of an aneurysm, the aneurysm itself may be filled with the mechanical devices which cause formation of an emboli and, at some later time, is at least partially replaced by neovascularized collagenous material formed around the implanted devices.

A selected site is reached through the vascular system using a collection of specifically chosen catheters and/or guide wires. It is clear that should the site be in a remote site, e.g., in the brain, methods of reaching this site are somewhat limited. One widely accepted procedure is found in U.S. Pat. No. 4,994,069 to Ritchart, et al. It utilizes a fine endovascular catheter such as is found in U.S. Patent No. 4,739,768, to Engelson. First of all, a large catheter is introduced through an entry site in the vasculature. Typically, this would be through a femoral artery in the groin. Other entry sites sometimes chosen are found in the neck and are in general well known by physicians who practice this type of medicine. Once the introducer is in place, a guiding catheter is then used to provide a safe passageway from the entry site to a region near the site to be treated. For instance, in treating a site in the human brain, a guiding catheter would be chosen which would extend from the entry site at the femoral artery, up through the large arteries extending to the heart, around the heart through the aortic arch, and downstream through one of the arteries extending from the upper side of the aorta. A guidewire and neurovascular catheter such as that described in the Engelson patent are then placed through the guiding catheter as a unit. Once the distal end of the catheter is positioned at the site, often by locating its distal end through the use of radiopaque marker material and fluoroscopy, the catheter is cleared. For instance, if a guidewire has been used to position the catheter, it is withdrawn from the catheter and then the assembly, for example including the implantable device at the distal end, is advanced through the catheter. The device is advanced past the distal end of the catheter so that it is free and positioned precisely at the desired treatment site.

The length of delivery mechanism will be such as to be capable of being advanced entirely through the catheter to place implantable device at the target site but yet with a sufficient portion of the distal end of the delivery mechanism protruding from the distal end of the catheter to enable detachment of the implantable device. For use in peripheral or neural surgeries, the delivery mechanism will normally about 100-200 cm in length, more normally 130-180 cm in length. The diameter of the delivery mechanism is usually in the range of 0.25 to about 0.90 mm.

Once the implantable device is at the selected site, the desired junction point is selected and the appropriate wavelength of electromagnetic radiation (e.g., light) is then supplied by the energy source and transmitted through the delivery mechanism to the selected junction. The selected junction is sufficiently melted so as to free the device from the deployment mechanism and/or rest of the device at that junction. This procedure can be repeated as desired. Following severing of the selected junction(s), the entire catheter may then be removed or the delivery mechanism may be withdrawn from the catheter lumen to provide for installation of other implantable devices. If additional implants are to be placed at the target site, the procedure is repeated. After the desired number of implants have been placed at the site, the catheter is withdrawn from the vessel.

If it is desired to further protect the device from heating effects during detachment, insulating materials may be included in the device between one or more of the junction sites. If such an additional insulating member is used, it is desired, but not necessary, that it consist of an electrically insulating polymer material and/or thickness different from that of the thermoplastic member such that the thermoplastic member preferentially absorbs the energy applied during detachment by the energy source. The insulating material can be a polymer such as polyethylene, polypropylene, polyurethane, polyethylene terephthalate, polyvinylchloride, and is preferably a polymer from the class of polymers generally known as parylene. The insulation may be applied to the proximal end of delivery mechanism by a number of processes such as shrink-wrapping, dipping in molten polymer, spraying on in the form of a suspension or latex, or the like. The axial length of the additional insulating member and its thickness may vary depending upon the degree of additional electrical insulation desired, the specific configuration of the assembly, the application for which assembly is used, etc.

Modifications of the procedure and device described above, and the methods of using them in keeping with this invention will be apparent to those having skill in this mechanical and surgical art. These variations are intended to be within the scope of the claims that follow.

What is claimed is:

1. An implantable device comprising a plurality of detachment junctions, wherein each junction is cleaved by the application of a different wavelength of electromagnetic radiation.

2. The device of claim 1, wherein the electromagnetic radiation is light.

3. The device of claim 1, wherein one or more junctions comprise a shape memory polymer.

4. The device of claim 2, wherein one or more junctions further comprise one or more dyes or pigments.

5. The device of claim 1, wherein the implantable device comprises a vaso-occlusive coil.

6. The device of claim 1, wherein the implantable device comprises a stent.

7. The device of claim 1, wherein the implantable device comprises a filter.

8. The device of claim 2, wherein the light is visible light.

9. The device of claim 2, wherein the light is non-visible light.

10. An assembly for use in delivering an implantable device comprising
(a) an implantable device according to claim 1; and
(b) a deployment mechanism.

11. The assembly of claim 10, wherein the deployment mechanism comprises one or more electromagnetic radiation transmitting devices.

12. The assembly of claim 11, wherein the electromagnetic radiation transmitting device comprises one or more fiber optic cables.

13. The assembly of claim 11, wherein the electromagnetic radiation transmitting device comprises one or more light-transmitting fluids.

14. The assembly of claim 11, wherein the electromagnetic radiation transmitting device comprises one or more light-transmitting wires.

15. The assembly of claim 11, wherein the implantable device comprises a vaso-occlusive coil.

16. The assembly of claim 11, wherein the implantable device comprises a stent.

17. The assembly of claim 11, further comprising (d) a source of electromagnetic radiation attached to the delivery mechanism.

18. The assembly of claim 17, wherein the electromagnetic radiation is light.

19. The assembly of claim 18, wherein the light source comprises a laser.
